(19) United States
(12) Patent Application Publication
(10) Pub. No.: US 2013/0325140 A1
(43) Pub. Date: Dec. 5, 2013

(54) DEVICE FOR PLACEMENT IN A HOLLOW ORGAN, IN PARTICULAR FOR HOLDING OPEN SAID HOLLOW ORGAN AND METHOD FOR PRODUCING SUCH DEVICE

(71) Applicants: Frank Willems, Moers (DE); Christoph Classen, Houschian (DE); Andreas Henseler, Simmerath (DE); Wolfgang Witt, Moers (DE)

(72) Inventors: Frank Willems, Moers (DE); Christoph Classen, Houschian (DE); Andreas Henseler, Simmerath (DE); Wolfgang Witt, Moers (DE)

(73) Assignee: NonWo Tecc Medical GmbH, Cologne (DE)

(21) Appl. No.: 13/701,620

(22) PCT Filed: May 31, 2011
(86) PCT No.: PCT/EP2011/058931

§ 371 Date: Jul. 25, 2013

Related U.S. Application Data
(60) Provisional application No. 61/433,400, filed on Jan. 17, 2011.

(30) Foreign Application Priority Data
Jun. 2, 2010 (EP) 10164721.2

(51) Int. Cl. A61F 2/90

(52) U.S. Cl. CPC A61F 2/90
USPC 623/23.7; 427/2.25

(57) ABSTRACT

The device for placement in a hollow organ, in particular for holding open the hollow organ, comprises a placement body (12) having an inner side and an outer side (14). The device further comprises at least one layer (16) of biostable random-fiber fleece material arranged on said placement body (12) and being at least partially in abutment thereon.

The present invention relates to a device for placement in a hollow organ, in particular for holding open the hollow organ, and a method for producing such a device.

In an exemplary application, the device for placement in a hollow organ is a vascular support for holding open blood vessels, which herein will be mentioned as an example of a (biological) hollow organ, which is known inter alia under the term “stent”. Vascular prostheses, on the other hand—in contrast to vascular supports which serve for holding open e.g. a blood vessel and thus do not replace the blood vessel and respectively the hollow organ—are medical products provided for replacement of biological tissues.

The invention will be explained hereunder, by way of example, with reference to a vascular support for a blood vessel embodied as a placement device for hollow organs, wherein, first, the problematics of presently known vascular supports will be outlined.

The clinical picture of arteriosclerosis is characterized by a pathological deposition of various substances—inter alia calcium and fat—on the wall of a blood vessel (arteriosclerotic plaque). The resultant narrowing of the inner vessel diameter causes a reduced passage for the blood flow, which in turn will result in an undersupply to the tissue downstream of this site.

The above narrowing of vessels can be clinically reversed by means of a vascular support (stent), which is effected by mechanically widening the vessel wall and keeping it in this state by placement of the stent. However, after such an intervention, it frequently happens that a renewed narrowing develops at the given site because the vessel wall may proliferate into the vessel lumen through the interspaces of the stent lattice.

In case of an instable plaque, only a small spatial delimitation exists between the substances deposited on the vessel wall and the interior of the vessel (blood flow). In such a case, a plaque rupture may occur so that the deposited substances will enter the blood flow. In the periphery, these substances can cause occlusions or initiate thrombotic occlusions. This risk is further increased by use of non-coated vascular supports in arteriosclerotic vessels covered by instable, inelastic plaque because, at such sites, the dilatation of the vascular support will cause an increased mechanical stress acting on the inner wall of the vessel, which in turn will heighten the risk of rupture.

In a further type of clinical application, vascular supports are used for treatment of aneurysms in vessels. In this case, use is made of special vascular supports whose wall is formed by a closed material, normally, a PTFE of little elasticity. These vascular supports have to be implanted with the aid of a complex applicator in order to allow them to be converted, within the vessel, from a folded state to an expanded state and to be placed in position. This process entails the risk that folds may be generated in cases where it had not been possible to accomplish an optimal adaptation of the vascular support diameter to the vessel diameter.

From DE 102 23 399 B4, there is known a vascular support, provided for supporting a vessel wall, which comprises mutually adjacent support elements and a film of absorbable material enclosing said support elements, wherein said film can consist of a nonwoven. Said film or fleece has the function of keeping the individual support elements at a distance until, over time, they will have become fixed to the vessel wall. Said film or fleece will be absorbed so that, of the overall vascular support, it is only the support elements which will ultimately remain in the vessel.

Known from DE 10 2006 028 221 A1 is a hose-shaped vascular prosthesis comprising a wall with an elastic fleece structure which under physiological conditions is substantially non-absorbable. The vascular prosthesis can be provided with reinforcement elements. As already mentioned above, the vascular prosthesis serves for replacement of biological tissue and not for the purpose of maintaining it in an open state or for the purpose of supporting hollow organs. Due to this intended use, it is not possible in vascular surgery to use prostheses for maintaining hollow organs in an open state because the prostheses, according to their purpose, shall replace the hollow organs and thus, logically, cannot support a hollow organ while the latter remains in place.

Another prosthesis, namely an end prosthesis, is described in U.S. Pat. No. 5,549,663. This prosthesis comprises a stent component and a graft component capturing a part of the stent component. Said graft component consists of helically wound fibers by which the thus tubular graft component is given an axial and thus anisotropic and respectively one-dimensional preferred stretching direction.

It is an object of the invention to provide a device for placement in a hollow organ wherein the risk of the above mentioned complications during and after implantation is reduced.

According to the present invention, the above object is achieved by a device for placement in a hollow organ, which comprises
a placement body having an inner side and an outer side, and
at least one layer of biostable random-fiber fleece material arranged at said placement body and at least partially abutting thereon.

The invention further proposes a method for producing said device wherein, according to said method, there is first provided said placement body and, for positioning of said layer of biostable random-fiber fleece material, the placement body is sprayed with fibers by use of a spraying device while the placement body and the spraying device are moved relative to each other, and wherein, in dependence on the desired thickness of said layer of biostable random-fiber fleece material, a plurality of layers of fibers are sprayed on.

In essence, the invention resides in that the placement body for placement in a hollow organ will be provided with a biostable fleece material comprising interconnected and, particularly, fine-fibrillated fibers made e.g. of polyethylene. In this arrangement, the biostable random-fiber fleece material (hereunder referred to merely as a fleece material) is at least partially in abutment on the inner side and/or on the outer side of the placement body of the device.

The following is a description of the invention with reference to its application as a vascular support for holding open a hollow organ, wherein the placement body is referred to as a support body.

Said fleece material, which is biostable, i.e. under physiological conditions is substantially not absorbable, preferably comprises a fine-fibrillated fleece whose fibers are connected to each other. Particularly, this fleece material can have such a porosity that the liquid components of the blood or of the substances taken up by the hollow organ will be substantially allowed to pass while, however, the cellular components of the blood or of the substances taken up by the hollow organ and of the vessel wall will be substantially retained. This feature advantageously allows for a complete exchange of liquids and chemical elements, particularly of nutrients, metabolites and other physiological substances between the inner fluid and the vessel wall. By the random-fiber fleece material of the invention, the support body can be stretched in an isotropic manner, which is of advantage for a multi-dimensional supporting function that is effective in a plurality of spatial directions.

According to a further advantageous embodiment of the invention, it is provided that the fleece material layer in the area of the inner surface has a porosity which is smaller than the porosity on the outer surface of the fleece material layer. When viewed in the thickness direction of the fleece material layer, the porosity of the fleece material can vary in a continuous, quasi-continuous or step-wise manner.

According to a further advantageous embodiment of the invention, the bio-stable fleece material, on its inner surface which comes into contact with the substances taken up by the hollow organ, is capable of rendering possible, or facilitating, the adherence and colonization of blood cells, stem cells, progenitor cells or blood-vessel wall cells or, more generally, of cells of the substances taken up by the hollow organs. This is achieved particularly by corresponding selection of the porosity of the fleece material on the surface thereof which comes into contact with the tissue, and of the porosity at that site of the fleece material layer which is in connection with the substance taken up by the hollow organ. The porosity at said above described surface of the fleece material is selected to the effect that the connective tissue proliferating from the hollow organ cannot penetrate the fleece material layer. Thereby, the risk that the hollow organ might become clogged or overgrown later on, is minimized.

According to a further advantageous embodiment of the invention, the fleece material layer comprises an outer surface and an inner structure which renders possible or facilitates the integration of the connective tissue.

Finally, it is of advantage if the fleece material layer is tightly connected with the support body of the vascular support. This is suitable primarily because, in this manner, the position of the fleece material layer relative to the support body will not be changed during the implanting of the vascular support as well as subsequently, i.e. in situ.

According to a further advantageous embodiment of the invention, it is provided that the elasticity and the porosity of the fleece material are adjusted in such a manner that the desired pore size for promoting a well-aimed cell migration will be obtained only after a possible intended dilatation of the vascular support. The inventive biostable fleece material will expand corresponding to the dilatation of the support body, as far as such a preferably permanently expandable support body is used in the inventive vascular support. In such a vascular support, it is of advantage if, in the dilated state, the pore size of the fleece material has the desired value or is in the desired range of values. On the basis of the degree of dilatation and the properties of the fleece material used, it can be determined, by backward calculation, which pore size the fleece material should have in the not-yet-dilated state of the support body in order to accomplish a desired pore size or range of pore sizes in the fleece material and respectively in its surfaces. In any case, the elasticity of the fleece material has to be provided to the effect that the dilated support body cannot be squeezed together again by the widened fleece material.

The inventive arrangement of a layer of biostable fleece material with at least partial abutment on the support body of a vascular support surprisingly leads to a lower postoperative complication rate after implantation of a vascular support for the purpose of vessel dilatation. Both the risk of plaque rupture and the consequences of such a plaque rupture due to the placement of a vascular support are considerably reduced by using a fleece material layer on the support body. In this manner, an occlusion of peripheral vessels is prevented.

According to a further advantageous embodiment of the invention, it is provided that the biostable fleece material completely covers the outer side and/or the inner side of the support body.

A further functional advantage of the invention can be seen in the feature that the enclosure does not represent a compactly closed structure but instead consists of a three-dimensional, microporous, fine-fibrillated fiber structure. Thereby, the physiology of the vessel wall is not restricted as much as when using dense, closed materials. Thus, this material structure allows for an exchange of substances from and to the vessel wall and also offers the possibility of a selective adhesion, migration and proliferation of cells. The result is the generation of an endothelium-like layer which can be formed toward the blood.

By the differentiated configuration of the fleece structure, a well-aimed colonization of cells is achieved. Thereby, the vascular support will become completely fixed in position by integrative healing, so that the inventive product can be conceived of as a catalyst for the reestablishment of physiological conditions on the vessel wall.

The invention described herein is applicable in biological vessel systems, particularly in coronary vessels, peripheral vessels (arterial and venous applications) and neurovascular vessels. Apart from these vessels, the hollow organs which can be kept open by the inventive vascular support also include lymph vessels, renal ducts, urethrae, the esophagus, nerve cords or uterine tubes.

In another application, the enclosed placement body as provided according to the invention can be used for therapy of vessel aneurysms of any type (fusiform, sacriform, pedunculated and non-pedunculated aneurysms). By the presence of the microporous fleece structure, there will first occur a stasis and a thrombosis formation in the aneurysmal sac. Subsequent wound healing processes with an absorption of the thrombus and a replacement of connective tissue will allow the aneurysm to heal. Also here, the later formation of a functional endothelium layer on the inner side of the implant will very quickly lead to laminar flow conditions in the area of the aneurysm. This physiological replacement for closure of the aneurysmal sac is safer and requires distinctly less time for the surgical intervention than is possible e.g. through the conservative method by filling with coils. By implanting the placement body the aneurysmal sac is bypassed so that blood flows through the lumen of the placement body, thereby preventing further ingress of blood into the aneurismal sac. Moreover, this closure of the aneurysmal sac prevents thrombogenesis in the blood vessel.

According to a still further embodiment, the invention is suited for use also in the non-vascular region, e.g. as a gastrointestinal stent. Also in this application, the promotion of a physiological cell proliferation by the fine-fibrillated fleece for thus forming a natural vessel-wall layer is of eminent advantage.

In case of an application for tumor diseases, the tumor tissue can hardly grow through the areal enclosure into the lumen.

The biostable fleece material of the device of the invention is suitably formed from an elastomer, preferably form a thermoplastic elastomer, With preference, the fleece structure is made of polyurethane, particularly linear polyurethane. With particular advantage, the polyurethane is an aliphatic polyurethane, preferably formed of macromolecular and/or low-molecular aliphatic diols as well as aliphatic diisocyanates. According to the invention, it is especially preferred that said macromolecular diols are polycarbonates, particularly 1,6-hexanediol polycarbonate. Said low-molecular diols preferably are 2,2,4-trimethylhexanediol, 2,4,4-trimethylhexanediol and/or 1,4-butanediol. Preferably, said aliphatic diisocyanates are 4,4′-dicyclohexylmethane diisocyanate or 1,4-cyclohexyl diisocyanate. According to the invention, it can further be preferred that said aliphatic polyurethane is formed of different diols and/or diisocyanates, wherein preference is given to the diols and diisocyanates described in this paragraph. Concerning further details and features of polyurethanes, reference is made to DE-A-36 43 465, DE-A-33 18 730, DE-A-41 07 284 and to the polymer report “Biocompatible Polyurethanes for Medical Techniques” of the research institute of Enka AG in Obernburg, wherein the disclosure of each of said documents is herewith, by way of reference, incorporated to its full extent into the present description.

According to the invention, the fleece material layer comprises fibers having a diameter from 0.1 μm to 100 μm, preferably from 0.2 μm to 20 μm and more preferably from 0.3 μm to 1 μm.

According to the arrangement of the invention, the fleece material layer has a bottom side which is in abutment on the outer side of the support body, and a top side facing away from the outer side of the support body, Further, the fleece material layer comprises, on its top side, pores of a size different from that of the pores on its bottom side. According to the invention, the pores on the bottom side of the fleece material layer are smaller than the pores on the top side of the fleece material layer. The ratio between the pore size on bottom side and the pore size on top side is 1:50, preferably 2:10 and more preferably 4:8.

The fleece material layer has a thickness from 10 μm to 3000 μm.

According to a preferred embodiment of the invention, the support body is expandable, particularly in a permanent manner, with the fleece material layer being stretched at the same time, wherein, prior to the expansion, the thickness of the fleece material layer is from 100 μm to 3000 μm, preferably from 150 μm to 2800 μm and more preferably between 200 μm and 2000 μm.

According to a further preferred embodiment of the invention, the support body is expandable, particularly in a permanent manner, with the fleece material layer being stretched at the same time, wherein, after the expansion, the thickness of the fleece material layer is from 10 μm to 2500 μm, preferably from 20 μm to 2000 μm and more preferably between 80 μm and 1000 μm.

According to the invention, the vascular support comprises a further layer of biostable fleece material, wherein the inner side and the outer side of the support body each comprise respectively one fleece material layer which is arranged at least partially in abutment on the respective side.

According to the invention, the support body is porous and particularly has a reticular structure.

In the inventive method for producing the vascular support, there can be performed e.g. the procedural steps described in the Applicant's not-yet-published PCT Patent Application PCT/EP2010/066928. According to these methods, whose features are herewith, by way of reference to the respective documents, incorporated into the present application, the fleece material is sprayed in the form of microfibers onto a rotating shaped member. According to the invention, said shaped member comprises the support body of the vascular support.

Preferably, in said method, there is first provided the support body and, with the aid of a spraying device, the support body is sprayed with fibers for thus applying the layer of biostable fleece material. In the process, the support body and the spraying device are moved relative to each other. In dependence on the desired thickness of the layer of biostable fleece material and/or the desired porosity, a plurality of fiber layers will be spray-deposited, optionally with different areal densities.

A full and enabling disclosure of the present invention, including the best mode thereof, enabling one of ordinary skill in the art to carry out the invention, is set forth in greater detail in the following description, including reference to the accompanying drawing in which

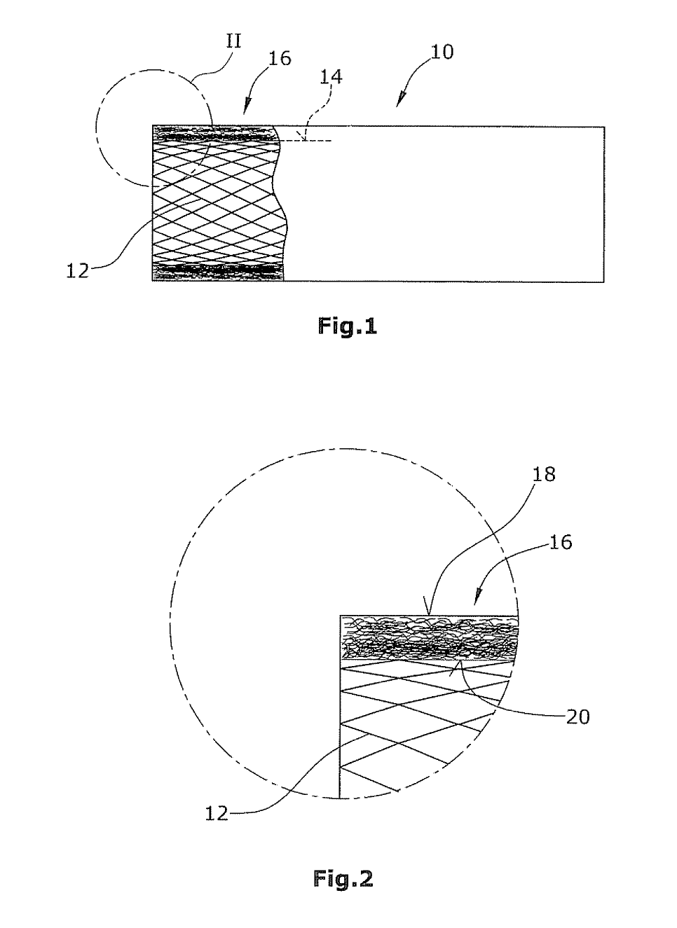

FIG. 1 is a lateral view of a tubular vascular support to be used in a blood vessel for holding open the blood vessel, wherein a part of the illustrated vascular support is broken away to visualize the wall structure of the vascular support, and

FIG. 2 is an enlarged view of the detail II in FIG. 1.

FIG. 1 shows, in lateral view, a tubular vascular support 10 (e.g. a stent) comprising a reticular or net-shaped support body 12 whose cylindrical outer side 14 is provided with an enclosure made of a layer 16 of biostable fleece material. Said fleece material layer 16 comprises a random-laid fleece made of microfibers. Fleece material layer 16 has a larger porosity on its outer side 18 than on its inner side 20. By its outer side 18, fleece material layer 16 is arranged in abutment on the vessel wall (not shown). As achieved by the invention, tissue proliferating from the vessel wall will only partially intrude into the fleece material layer 16. Such a proliferation of the tissue will be stopped at the latest in that region of the fleece material layer 16 which is located near the support body 12, particularly on the inner side 20 of layer 16 whose pore size is selected to the effect that a further proliferation of tissue through the fleece material layer 16 will not be possible anymore.

1. A device for placement in a hollow organ, in particular for holding open said hollow organ, comprising:
a placement body having an inner side and an outer side, and
at least one layer of biostable random-fiber fleece material arranged at said placement body and at least partially in abutment thereon.

2. The device according to claim 1, wherein said random-fiber fleece material layer is at least partially arranged in abutment on said outer side of the placement body.

3. The device according to claim 2, wherein the random-fiber fleece material layer comprises fibers having a diameter from 0.1 μm to 100 μm, preferably from 0.2 μm to 20 μm and more preferably from 0.3 μm to 1 μm.

4. The device according to claim 2, wherein the random-fiber fleece material layer has a bottom side which is in abutment on the outer side of the placement body, and a top side facing away from the outer side of the placement body, and wherein the random-fiber fleece material layer on its top side comprises pores of a size different from that of the pores on its bottom side.

5. The device according to claim 4, wherein the pores on the bottom side of the random-fiber fleece material layer are smaller than the pores on the top side of the random-fiber fleece material layer.

6. The device according to claim 4, wherein the ratio between the pore size on bottom side and the pore size on top side is 1:50, preferably 2:10 and more preferably 4:8.

7. The device according to claim 1, wherein the random-fiber fleece material layer has a thickness from 10 μm to 3000 μm.

8. The device according to claim 1, wherein the placement body is particularly permanently expandable, with the random-fiber fleece material layer being stretched at the same time, and wherein, prior to expansion, the thickness of the random-fiber fleece material layer is from 100 μm to 3000 μm, preferably from 150 μm to 2800 μm and more preferably between 200 μm and 2000 μm.

9. The device according to claim 1, wherein the placement body is particularly permanently expandable, with the random-fiber fleece material layer being stretched at the same time, and wherein, after expansion, the thickness of the random-fiber fleece material layer is from 10 μm to 2500 μm, preferably from 20 μm to 2000 μm and more preferably between 80 μm and 1000 μm.

10. The device according to claim 1, wherein a further layer of biostable random-fiber fleece material is provided, the inner side and the outer side of the placement body each comprising respectively one random-fiber fleece material layer arranged at least partially in abutment thereon.

11. The device according to claim 1, wherein the placement body is porous and particularly has a reticular structure.

12. A method for producing a device according to claim 1, wherein the placement body is provided and, with the aid of a spraying device, the placement body is sprayed with fibers for thus applying said layer of biostable random-fiber fleece material, while the placement body and the spraying device are moved relative to each other, and wherein, in dependence on the desired thickness of the layer of biostable random-fiber fleece material, a plurality of fiber layers are spray-deposited.
